(19) United States
(12) Patent Application Publication
(10) Pub. No.: US 2021/0052331 A1
(43) Pub. Date: Feb. 25, 2021

(54) VERSATILE IMAGING

(71) Applicant: Navix International Limited, Road Town (VG)

(72) Inventor: Shlomo BEN-HAIM, Milan (IT)

(73) Assignee: Navix International Limited, Road Town (VG)

(21) Appl. No.: 17/089,799

(22) Filed: Nov. 5, 2020

Related U.S. Application Data
(63) Continuation of application No. PCT/IB2019/053677, filed on May 6, 2019.
Application No. PCT/IB2019/053677 is a continuation-in-part of application No. PCT/IB2018/059672, filed on Dec. 5, 2018.
(60) Provisional application No. 62/777,817, filed on Dec. 11, 2018.
(60) Provisional application No. 62/667,653, filed on May 7, 2018.
(60) Provisional application No. 62/667,653, filed on May 7, 2018.

(51) Int. Cl. A61B 34/20; A61B 5/053; A61B 17/3211; A61B 17/34

(52) U.S. Cl. CPC A61B 34/20; A61B 5/0536; A61B 2034/2072; A61B 17/3403; A61B 2034/2053; A61B 17/3211

(57) ABSTRACT

Electrical field imaging is performed using optionally any of a range of medical implements having a designated medical use (e.g., having a working portion for manipulating tissue); and furthermore supplied with at least one electrode that is used to transmit and/or receive an electrical field which interacts with the dielectric properties of tissue. In some embodiments, the at least one electrode is supplied as an add-on assembly, optionally as part of a kit including the medical implement.

### RELATED APPLICATIONS

This application is a Continuation of PCT Patent Application No. PCT/IB2019/053677 having International filing date of May 6, 2019, which claims the benefit of priority under 35 USC § 119(e) of U.S. Provisional Patent Application Nos. 62/777,817 filed on Dec. 11, 2018 and 62/667,653 filed on May 7, 2018.

PCT Patent Application No. PCT/IB2019/053677 is also a Continuation-in-Part (CIP) of PCT Patent Application No. PCT/IB2018/059672 having International filing date of Dec. 5, 2018, which claims the benefit of priority under 35 USC § 119(e) of U.S. Provisional Patent Application No. 62/667,653 filed on May 7, 2018.

The contents of the above applications are all incorporated by reference as if fully set forth herein in their entirety.

### FIELD AND BACKGROUND OF THE INVENTION

The present invention, in some embodiments thereof, relates to the field of imaging; and more particularly, to imaging used for the guidance of medical procedures.

In many interventional medical procedures, the operator can benefit by having the ability to real-time image the anatomy in the relevant vicinity of the action field of the procedure. In cardiac electrophysiology mapping in particular (e.g., for cardiac electrophysiology ablation as well as in other interventional cardiac procedures), multiple technologies exist for providing real-time imaging of the action field.

Intra-procedure imaging technologies include those that can image remotely from a chamber wall, and those that can reconstruct a chamber wall using the knowledge of the location of multiple points on the wall or multiple points within the chamber, or a combination of the last two.

### SUMMARY OF THE INVENTION

There is provided, in accordance with some embodiments of the present disclosure, a method of imaging using movements of a medical implement comprising a working portion and at least two electrodes affixed to the medical implement, wherein the working portion is configured to manipulate an internal anatomical structure upon reaching a position of mechanical contact therewith, the method comprising: transmitting an electrical field from at least one transmitting electrode to interact with an internal anatomical structure; moving the working portion of the medical implement among positions away from the position of mechanical contact, while the transmitted electrical field interacts with the internal anatomical structure; receiving the electrical field by at least one receiving electrode; measuring changes in the electrical field due to the moving and the interacting with the internal anatomical structure; and constructing an image of the internal anatomical structure, based on the measuring; wherein the at least two electrodes affixed to the medical implement comprise at least one of the at least one transmitting electrode and the at least one receiving electrode.

In some embodiments, the at least two electrodes comprise both the at least one transmitting electrode and the at least one receiving electrode.

In some embodiments, the at least two electrodes comprise an electrode covering a surface of the working portion.

In some embodiments, the method comprises moving the working portion to the position of mechanical contact, based on the position of the working portion relative to a position of the internal anatomical structure shown in the image.

In some embodiments, the method comprises displaying the image to a user, along with a representation of the working portion at a current position relative to the internal anatomical structure.

In some embodiments, the constructing comprises estimating a composition of tissue in the region of the anatomical structure, based on the measuring.

In some embodiments, the estimating is based on estimating a distribution of dielectric properties in the region of the anatomical structure which accounts for changes measured.

In some embodiments, the constructing comprises associating particular positions of the at least one electrode to the changes measured in the electrical field due to the moving and the interacting.

In some embodiments, the associating particular positions is based on at least one distance between a plurality of electrodes of the at least one electrode.

In some embodiments, the at least one electrode comprises three or more electrodes, each at a known distance from another of the three or more electrodes.

In some embodiments, the medical implement is held at a handle which controls positioning of the working portion, and the handle and the working portion are rigidly interconnected.

In some embodiments, the medical implement is held at a handle which controls positioning of the working portion, and the handle and the electrodes are rigidly interconnected.

In some embodiments, the medical implement is a scalpel.

In some embodiments, the working portion is moved by manipulation of the medical implement while holding the medical implement within 20 cm of the working portion.

In some embodiments, the working portion comprises a blade, configured to cut tissue.

In some embodiments, the working portion comprises a sharpened tip, configured to pierce tissue.

In some embodiments, the working portion comprises a sharpened portion, configured to dissect tissue.

In some embodiments, the positions away from the position of mechanical contact are separated from the internal anatomical structure by a thickness of solid tissue.

In some embodiments, the thickness of solid tissue is at least 1 cm thick.

In some embodiments, the positions away from the position of mechanical contact are outside an exterior surface of an organ comprising the internal anatomical structure.

In some embodiments, the positions away from the position of mechanical contact are external to a cardiovascular lumen.

There is provided, in accordance with some embodiments of the present disclosure, a method of modifying and using a medical implement having a designated use, the method comprising: attaching at least one electrode to the medical implement; using the medical implement for its designated use, while the at least one electrode transmits and/or receives an electrical field modified by intersection with tissue; guiding movement of the medical implement, based on an image of the tissue reconstructed based on measurements of the electrical field transmitted and/or received by the at least one electrode.

In some embodiments, the at least one electrode transmits, and at least one additional measuring electrode is provided, positioned to sense changes in the electrical field during the using the medical implement.

In some embodiments, the at least one electrode receives, and at least one additional transmitting electrode is provided, positioned to transmit the electrical field during the using the medical implement.

In some embodiments, the movement is guided by the image and a position of the medical implement relative to the image.

There is provided, in accordance with some embodiments of the present disclosure, a system augmenting a medical implement, comprising: the medical implement, wherein the medical implement comprises a body terminating in a rigid distal end, the rigid distal end being at least 5 cm in length, and at least one electrode within 3 cm from a distal-most tip of the rigid distal end; and circuitry configured to send and/or receive electrical signals via the at least one electrode and provide an image of tissue adjacent the tip therefrom.

There is provided, in accordance with some embodiments of the present disclosure, an imaging system comprising: at least one electrode configured for attachment to a tool surface, or attached to a tool surface; wherein the electrode is not used for a medical interaction with a body part; signal circuitry configured to send and/or receive electrical signals from the electrode; and reconstruction circuitry configured to reconstruct an image from the signals, the image including a representation of the tool based on the signals.

There is provided, in accordance with some embodiments of the present disclosure, a kit for an imaging system comprising: a medical implement; at least one electrode attachable onto a surface of the medical implement; wherein the at least one electrode is configured for: transmitting an electrical field, and measuring changes in the electrical field due to movements of the medical implement which modify interactions of the electrical field with an internal anatomical structure; and a communication channel, configured for communication of the received indications to reconstruction circuitry configured to reconstruct an image from the received indications.

In some embodiments, the at least one electrode is attachable to the surface by fitted attachment of a sleeve comprising the at least one to the medical implement.

In some embodiments, the at least one electrode is attachable to the surface by adhesion to the medical implement.

There is provided, in accordance with some embodiments of the present disclosure, a guidewire for an imaging system comprising: a catheter guidewire; at least one electrode integrated with the guidewire; wherein the at least one electrode is configured for: transmitting an electrical field, and receiving the electrical field, including receiving changes in the electrical field due to interactions of the electrical field with an internal anatomical structure; and a communication channel, configured for communication of the received signals to reconstruction circuitry configured to reconstruct an image from the received changes.

There is provided, in accordance with some embodiments of the present disclosure, a method of imaging using a tool comprising a working portion configured to manipulate tissue of a body, and at least one electrode affixed to the tool, the method comprising:

transmitting electrical signals through the tissue while the working portion and at least one electrode move together; sensing the electrical signals transmitted through the tissue using the at least one electrode; and imaging the tissue, based on the sensing.

There is provided, in accordance with some embodiments of the present disclosure, a method of imaging using a medical implement comprising a working portion configured to manipulate tissue of a body, and at least one electrode affixed to the medical implement, the method comprising: transmitting electrical signals from the at least one electrode while the working portion and at least one electrode move together so that the electrical signals transmit through the tissue; sensing the electrical signals transmitted through the tissue using the at least one electrode; imaging the tissue, based on the sensing, to produce an image comprising a plurality of distinguishable features; and determining a location of the working portion, relative to locations of the plurality of distinguishable features.

There is provided, in accordance with some embodiments of the present disclosure, a method of imaging during a medical procedure, the method comprising: providing at least one electrode affixed to a medical implement used in the medical procedure to perform a tissue manipulation; transmitting electrical signals from the at least one electrode; and imaging using movements of the medical implement performed at least in preparation for the tissue manipulation, wherein the movements move the at least one electrode so that the electrical signals transmit through the tissue, the imaging comprising: sensing the electrical signals transmitted through the tissue using the at least one electrode; and producing an image comprising a plurality of distinguishable features; based on the sensing.

There is provided, in accordance with some embodiments of the present disclosure, a method of imaging comprising: imaging an internal body region using measurements from a first tool, wherein the measurements are measurements of an electrical field modified by features of the internal body region as the first tool moves outside the internal body region; and selecting a position of the internal body region, based on the imaging; and inserting a portion of the first tool into the internal body region at the selected position.

In some embodiments, the measurements are measurements of an electrical field modified by features of the additional internal body region as the second tool moves outside the internal body region.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable memory to receive instructions and/or data therefrom, process them, and/or store processing results in the same or another computer readable storage memory. The processing performed (optionally on the data) is specified by the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

### BRIEF DESCRIPTION OF THE SEVERAL VIEWS OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIG. 1 is a block diagram of a system for electrical mapping of an anatomical portion using a medical implement (as well as doctor and patient), according to some embodiments of the present disclosure;

FIG. 2 is a flowchart of a method of electrical-measurement based imaging of a body structure, according to some embodiments of the present disclosure;

FIGS. 3A-3B schematically illustrate a medical implement (a scalpel) approaching tissue including a target feature for imaging (which may represent, for example a tumor), according to some embodiments of the present disclosure;

FIG. 4 is a flowchart describing creation and use of an electrical field image as part of an overall procedure, according to some embodiments of the present disclosure;

FIGS. 5A-5D show cross-sections of liver images produced by simulation of electrical field measurements made during simulated movements of an electrode-equipped scalpel moving within an electrical field while measuring that electrical field, according to some embodiments of the present disclosure;

FIG. 5E shows relative positions of a scalpel and a liver (for example, as used in the simulations of FIGS. 5A-5D), and the plane of the image of FIGS. 5C-5D, according to some embodiments of the present disclosure;

FIG. 6A schematically illustrates a needle (e.g., a biopsy needle and/or an ablation needle) for use in a medical procedure, and provided with two electrodes, according to some embodiments of the present disclosure;

FIG. 6B is a flowchart describing a procedure for use of needle in a medical procedure, according to some embodiments of the present disclosure;

FIG. 7A schematically illustrates an adhesive electrode assembly, for use by attachment to a medical implement, according to some embodiments of the present disclosure;

FIG. 7B schematically illustrates a sleeve-attached electrode assembly, for use by attachment to a medical implement, according to some embodiments of the present disclosure;

FIG. 7C is a flowchart describing a method of attaching electrodes to a medical implement and calibrating the resulting implement for use in electrical field imaging, according to some embodiments of the present disclosure;

FIG. 8 schematically illustrates a laparoscope provided with a plurality of electrodes, according to some embodiments of the present disclosure;

FIGS. 9A-9C schematically illustrate stages in the insertion to a body of an electrode-equipped guide-wire configured for electrical imaging, according to some embodiments of the present disclosure;

FIG. 10 schematically illustrates an electrode-equipped guidewire configured for electrical imaging, shown in relation to an at least partially obstructed blood vessel, according to some embodiments of the present invention.

FIG. 11 schematically illustrates a guidewire equipped with electrodes, according to some embodiments of the present disclosure;

FIG. 12 is a flowchart describing use of a guidewire for imaging, according to some embodiments of the present disclosure; and

FIG. 13 is a flowchart describing use of electrode-based imaging tools during a medical procedure, according to some embodiments of the present disclosure.

### DESCRIPTION OF SPECIFIC EMBODIMENTS OF THE INVENTION

The present invention, in some embodiments thereof, relates to the field of imaging; and more particularly, to imaging used for the guidance of medical procedures.

### Overview

An aspect of some embodiments of the present disclosure relates to a method of imaging body structures using a suitably modified tool (medical implement) which is normally used for non-imaging and/or tissue manipulating purposes in a medical procedure.

In some embodiments, the medical implement is of a prior-known type having a designated use (e.g., a designated medical use), and modified from this prior-known type by manufacture with and/or by having added to it at least one electrode configured to be connected to a controller that operates the electrode to transmit and/or receive an electrical field which is modified by interaction with an imaged body region, according to the arrangement of dielectric properties of different regions within the imaged body region.

In some embodiments, the medical implement comprises, for example, a scissors, scalpel, knife, cannula, clamp, needle, syringe, laparoscope, guidewire, or another tool having a designated use (e.g., a designated medical use). In some embodiments, the medical implement comprises a working portion used for performing a tissue manipulation (e.g., a blade portion, other sharpened portion such as a tip, and/or tissue gripping portion). In some embodiments, the medical implement comprises a handle, rigidly interconnected with the working portion. In some embodiments, the shortest distance between the handle and the working portion is, for example, 40 cm or less, 30 cm or less, 20 cm or less, 10 cm or less, or another distance. In some embodiments, the medical implement comprises a body terminating at a rigid distal end (e.g., a distal-most tip), said rigid distal end being at least 4 cm, 5 cm, 6 cm, 7 cm, 8 cm, 9 cm, or more in length; and at least one electrode within 2 cm, 3 cm, 4 cm, 5 cm or another distance from said rigid distal end.

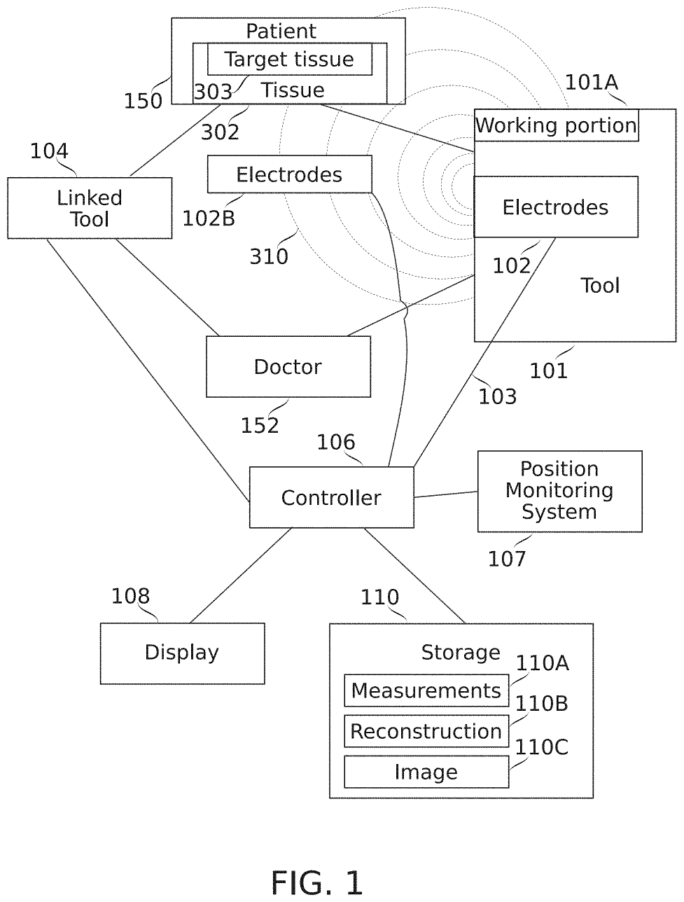

Other properties and/or types of medical implements used with the method in some embodiments are described, for example, in relation to tool 101 and FIG. 1.

In some embodiments, the method of imaging comprises transmitting a time-varying (e.g., radio frequency) electrical field so that the electrical field interacts with (and is affected by the dielectric properties of) an anatomical structure targeted to be imaged (within a target region). In some embodiments, the electrical field is transmitted using the at least one electrode. The method of imaging further comprises, in some embodiments, receiving by an at least one electrode (optionally an at least one electrode of the tool), electrical signals which indicate the interaction of the electrical field with the target anatomical structure. In some embodiments, the same at least one electrode of the medical implement is used to both transmit and receive the electrical field.

In some embodiments, the tool is moved within a sensing region while performing at least one (and optionally both) of transmitting and receiving an electrical field which interacts with the targeted anatomical structure. Along with the measuring, the position of the electrodes (optionally, a portion of the tool which has a determinable position with respect to the electrodes) is recorded. This results (optionally after a process of reconstruction) in a spatial map of an aspect of electrical field structure in a region of space substantially distinct (and optionally completely separate) from the target region. The electrical field structure is optionally a constant structure (e.g., when the field is transmitted from fixed-location electrodes). The electrical field structure is optionally a dynamic structure, dependent on a changing position from which the electrical field is transmitted. In some embodiments, the recorded measurements are doubly dynamic, e.g., measurements of a field that changes over time by being transmitted from different locations, and measured at different electrode positions.

In some embodiments, the process of reconstruction comprises using an approach such as multi-dimensional scaling (MDS) and local spatial coherence to estimate what the positions of the electrical measurements were, based on the measurements themselves, and optionally including one or more constraints such as knowing how far apart are two or more electrodes of the at least one electrode.

As the term is used herein, an “image” comprises a data structure separately representing at least two different locations of an imaged region according to their relative positions, wherein the imaged region comprises an arrangement of features (e.g., different living tissues) extending through some spatial extent. In some embodiments, the image distinguishes at least one difference among the at least two locations due to features that appear in the spatially extended arrangement of features.

The process of imaging, in some embodiments, comprises the process of constructing an image from measurement data. In some embodiments, the measurement data comprise measurements made using sensors (measuring electrodes, for example) in positions outside of the imaged region. In some embodiments, the measurements are made from positions also spatially separated from (i.e., “remote from”, as the term is used herein) the imaged region.

An aspect of some embodiments of the present disclosure relates to a method of modifying and using a medical implement configured for a designated medical use (that is, a use which for which the medical instrument is configured, even without the use of affixed electrodes as next described). The method, in some embodiments, includes affixing electrodes to the medical implement (e.g., in the form of an add-on electrode assembly augmenting the medical implement), and then using the medical implement for its designated medical use, while also using the electrodes to measure an electrical field which interacts with nearby tissue. In some embodiments, “nearby tissue” is tissue within a range of up to about 5 cm, 10 cm, 20 cm, or another distance. In some embodiments, the distance of the tissue being imaged from the medical implement (during imaging) is at least 1 cm, 5 cm, 10 cm, or another distance. The measurements are used to construct an image of a portion of the nearby tissue. In some embodiments, the tissue being imaged (nearby tissue) comprises at least two dielectrically distinct substances, e.g., different cellular types, and/or different tissue-defined structure (e.g., such as the structural differences between bone, lung, muscle and/or blood). Optionally, at least one of the dielectrically distinct substances comprises a target for a treatment or other interaction with the medical implement performing its designated medical use.

An aspect of some embodiments of the present disclosure relates to a kit for electrical field imaging, comprising a medical implement and at least one electrode configured to be affixed to the medical implement. The medical implement may be, for example a scissors, scalpel, knife, cannula, clamp, needle, syringe, laparoscope, or any other suitable medical implement. In some embodiments, the kit further comprises a communication channel (e.g., an electrical wire and/or a wireless communication device) for interconnecting between the at least one electrode and a controller, configured to make electrical measurements using signals received through the communication channel from the at least one electrode. Additionally or alternatively, the controller may be configured to transmit one or more electrical fields using the at least one electrode. In some embodiments, the kit comprises the controller.

An aspect of some embodiments of the present disclosure relates to an imaging guidewire comprising at least one electrode, and a communication channel configured to transmit signals received by the at least one electrode to a measuring device, for example, a controller.

An aspect of some embodiments of the present disclosure relates to use of electrode-based imaging tools during the course of a medical procedure. In some embodiments, electrical field imaging is performed at stages from the introducing stage of a catheter procedure through to electrical field imaging using the catheter and/or a guidewire of the catheter in a target body lumen such as a heart. During introduction, a catheter introduction needle (introducer or introducer needle) is optionally equipped with one or more electrodes and used as an electrical field imaging needle. Imaging optionally is used to locate an insertion target for the needle, which can then be inserted directly to the insertion target identified. Optionally, imaging continues during insertion, for example to confirm/help track the position of the needle, and/or to provide corrections, e.g., of an angle of introduction. In some embodiments, an electrical field imaging guidewire is then introduced through the needle. Optionally, electrical field imaging via the guidewire initiates in an area already imaged by the needle, and data from the guidewire imaging may continue to refine an image already produced during the location and insertion stages. The guidewire is guided along blood vessels to its target, optionally with electrical field imaging guidance, until it reaches its target, which may itself also be imaged by electrical field imaging. In some embodiments, this allows a whole catheter procedure to proceed without the use of X-ray radiation and/or contrast agent injection.

Before explaining at least one embodiment of the present disclosure in detail, it is to be understood that the present disclosure is not necessarily limited in its application to the details of construction and the arrangement of the components and/or methods set forth in the following description and/or illustrated in the drawings. Features described in the current disclosure, including features of the invention, are capable of other embodiments or of being practiced or carried out in various ways.

### Example Imaging System Using Medical Implement-Carried Electrodes

Reference is now made to FIG. 1, which is a block diagram of a system for electrical mapping of an anatomical portion using a medical implement (as well as doctor and patient), according to some embodiments of the present disclosure.

Tool 101 represents a medical implement (herein, the terms “tool” and “medical implement” are used interchangeably, except as noted) usable by a doctor 152 (optionally usable by hand-manipulation by a doctor 152). At least two electrodes 102 are affixed to tool 101. The at least two electrodes 102 are used for imaging of tissue 302 of patient 150 based on the effects of tissue 302 on measurements of one or more electrical fields 310 emitted by and/or received by the at least one electrode 102. It should be understood that doctor 152 and patient 150 (with target tissue 303) are shown in FIG. 1 for purposes of describing elements of the system and/or their operation; they are not part of the system itself.

In some embodiments, each electrode 102 comprises an electrode patch area of less than 5 mm², less than 4 mm², less than 2 mm², less than 1 mm², or another electrode patch area. Tool 101 itself may be one or both of electrically insulating and electrically conducting under each electrode 102, and/or insulating of conductive leads therefrom. Optionally, electrode 102 is separated from the main body of tool 101 by an added insulating material, for example, in embodiments where tool 101 comprises electrically conductive material under electrode 102

In some embodiments, tool 101 comprises, for example, a scissors, scalpel, knife, cannula, clamp, needle, syringe, laparoscope, guidewire, or another tool 101 having a designated medical use. Tool 101 optionally comprises one or both of rigid and flexible portions. In some embodiments, the designated medical use is a non-imaging use. Optionally, the designated medical use comprises manipulation of tissue 302 (e.g., an internal anatomical structure), for example, cutting, slicing, piercing, dissecting, grasping, suctioning, sampling, injecting, and/or holding tissue 302. Optionally, the designated medical use comprises guiding another tool into position to manipulate tissue 302. In some embodiments, the designated medical use comprises imaging using an optical method (for example, an optical camera), e.g., jointly with electrical field imaging. In some embodiments, the designated medical use comprises a use wherein a portion of the tool contacts and/or enters into a body of a patient. Herein, a tool having a designated medical use is also referred to as a tool for medically interacting with a body.

In some embodiments, tool 101 comprises a working portion 101A which directly performs the designated medical use, optionally while in mechanical contact with the tissue 302 (and optionally, more particularly a target feature 303 which is a tissue 303 which is to be imaged by electrical field imaging). In some embodiments, working portion 101A performs the designated medical use in line of sight of a target from a non-contacting distance, e.g., by delivering energy through plasma, laser light, or another energy form.

In some embodiments, electrodes 102 and working portion 101A are fixedly attached to each other (however, the fixed attachment is optionally temporary and/or reversible). For example, electrodes 102 are optionally attached to working portion 101A, e.g. in such a way as to cover a surface of working portion 101A. In some embodiments, electrodes 102 are attached to tool 101 (e.g., attached to cover a part of a surface of tool 101) in a position which is fixed relative to the position of working portion 101A. In some embodiments, electrodes 102 and working portion 101A are interconnected via the rest of tool 101 so that their relative position changes as a function of the operation of tool 101 (for example, working portion 101A may comprise a blade of a scissors, movable about a fulcrum relative to the position of electrodes 102 on another blade of the scissors).

In some embodiments, the at least two electrodes 102 comprises 2, 3, 4, 5, 6, 7, 8, and optionally more electrodes 102. Herein, it should be understood that embodiments shown and/or discussed for purposes of explanation in terms of any particular number of electrodes (e.g., 2 or 3 electrodes) are optionally provided with a different number of electrodes.

In some embodiments, the at least two electrodes 102 are operated in a self-sensing mode, wherein an electrical field transmitted from electrodes 102 is also received by electrodes 102 for making measurements. In some embodiments, electrodes are operated (e.g., supplied with a time varying current and/or voltage) in pairs to simultaneously transmit and receive electrical fields 310 which are affected by the dielectric environment through which the electrical fields 310 are transmitted. An electrode may be one or both of a transmitting electrode and a receiving electrode. In some embodiments where two or more electrodes transmit simultaneously, they transmit at different frequencies, so that each received signal can be associated (by frequency) with the electrode that transmitted it.

Additionally or alternatively, in some embodiments, there are optionally one or more additional electrodes 102B positioned elsewhere in the environment (that is, not fixedly attached to tool 101), e.g., attached to an outer skin surface of the patient, mounted to a surface of tissue 302, embedded in tissue 302, and/or mounted in a fixed position relative to tissue 302 (e.g., mounted to a table on which a patient is lying). Optionally, the additional electrodes 102B receive one or more electrical fields 310 transmitted from electrodes 102, and received signals are used as the basis of measurements of the one or more electrical fields 310. Additionally or alternatively, the additional electrodes 102B transmit one or more electrical fields 310 received by electrodes 102, and these received signals are used as the basis of measurements of the one or more electrical fields 310.

In some embodiments, transmitting and/or receiving by the at least one electrode 102 and/or at least one additional electrode 102B is performed under the control of a controller 106. In some embodiments, the at least one electrode 102 and controller 106 are in communication via a communication channel 103, to allow signals received by electrode 102 to be communicated to controller 106. In some embodiments, communication channel 103 comprises a cabled (e.g., electrical wiring and/or optical cabling) connection to controller 106 In some embodiments, communication channel 103 comprises a wireless segment of the communication channel 103, for example a segment over which communication is by radio, infrared, sonic, laser, or another transmitted energy. Controller 106, in some embodiments, is implemented at least partially as computer circuitry, e.g., comprising a digital data processor, digital memory, and digitally stored instructions (programmed instructions). In some embodiments, controller 106 comprise computer circuitry configured to reconstruct an image from the received signals (reconstruction circuitry).

Herein transmission of an electrical field 310 is also referred to as transmitting of a signal. Receiving and measuring are related insofar as a receiving electrode is connected to a measurement device which measures a parameter of the electrical field which is received. Accordingly, receiving of an electrical field may also be referred to herein as measuring the electrical field and/or measuring a signal transmitted through the electrical field (e.g., by the at least one electrode 102 operating under the control of a controller 106). In some embodiments, controller 106 comprises electrical measurement circuitry (e.g., comprising analog-to-digital sampling circuitry and/or signal amplification circuitry) for use in making measurements of electrical field signals received by electrodes 102 and/or electrodes 102B. In some embodiments, controller 106 comprises electrical signal generating circuitry (e.g., power conditioning, pulse generating, frequency selecting, and/or amplifying circuitry) used to drive electrical field transmission from electrodes 102 and/or 102B.

The effect of the nearby dielectric environment (and, in particular, dielectric properties of nearby tissue 302) on the electrical field 310 may result, for example, in an effect on the transmission of current to a receiving electrode 102, an effect on the transmission of a voltage to a receiving electrode 102, and/or in an effect on a phase at which the time-varying current and/or voltage is received at a receiving electrode 102. A measurement of voltage, current, and/or phase is an electrical measurement which may additionally or alternatively be used to calculate an impedance. The effect potentially changes depending on a position of a transmitting and/or receiving electrode 102 in relation to the nearby dielectric environment, including nearby tissue 302 and optionally one or more tissue targets 303 for electrical field imaging.

In some embodiments, an effect on a measurement by a receiving electrode 102 comprises an indication of a dielectric property of a region of tissue 302 through which the electrical field 310 is transmitted. In some embodiments, the frequency of time varying is a radio frequency. In some embodiments, the frequency of time varying is, for example, a frequency in the range from about 10 kHz up to about 10 MHz, for example, about 18.5 kHz.

In some embodiments, measurements received and/or processed by controller 106 are stored in measurement/map/image storage 110, comprising a suitable computer memory representation of electrical field measurements 110A, a reconstruction 110B of positions at which those electrical field measurements 110A were taken, and/or an electrical field image 110C of a region of tissue 302 constructed based on analysis of reconstruction 110B. Herein an “electrical field image” is an image created of a target region from measurements of an electrical field influenced by interactions with that target region (e.g., influenced due to the particular dielectric properties of different parts of the target region where the electrical field intersects those different parts of the target region). It should be understood that “electrical field” in this term references the imaging method, and is not describing the image as being per se an image of an electrical field. Optionally, the positions at which electrical field measurements 110A are obtained are determined from analysis of the measurements 110A themselves. This analysis is optionally made, e.g., in conjunction with constraining information such as known range of movements possible, the distances of the electrodes 102 from one another; and/or in conjunction with constraining assumptions on how the measurements could be distributed, such as an assumption that the measurements are spatially distributed in a manner that is “coherent” according to a chosen metric, e.g., a metric that constrains how quickly measurements can change as a function of distance and/or direction.

Additionally or alternatively, the positions at which electrical field measurements 110A are made are determined from a separate position monitoring system 107. For example, in some embodiments, position monitoring system 107 is part of a robotic positioning system used to control movements of tool 101, with the positions of electrodes 102 inferred from positions of the tool 101. In some embodiments, position monitoring system 107 is external to the electrical field transmitting and measurement system (e.g., a motion-tracking optical camera system). In some embodiments, electrodes 102 are themselves also sensing elements of a positioning monitoring system 107, for example a system that uses crossing electrical fields (optionally, but not necessarily the same electrical fields 310 used in imaging) to define a coordinate system, with measurements of the crossing electrical fields being used to establish positions within that coordinate system.

Controller 106, in some embodiments, is optionally in communication with a user interface including display 108, on which electrical field image 110C of tissue 302 derived from electrical field measurements may be displayed. Optionally, the electrical field image 110C is displayed together with a representation of tool 101 or a portion thereof. The representation may be displayed in the electrical field image 110C in a position which corresponds to its current position in space relative to the tissue 302 being imaged.

In some embodiments, a linked tool 104 is linked into the system in one or more ways. A linked tool may be any electrically transmitting tool used in a procedure that also uses tool 101 and might interfere with measurements made by electrode 102. Optionally, operation of the linked tool 104 gates at least one electrode 102, that is, the linked tool is configured to start and/or stop electrode 102. In some embodiments, the linked tool is gated by operation of the at least one electrode 102. In some embodiments, the gating is arranged to avoid mutual interference, e.g., electrical interference. In some embodiments, a position of linked tool 104 is tracked (e.g., by a position monitoring system 107), and its position can be shown in relation to electrical field image 110C.

### Examples of Use of an Imaging System Using Medical Implement-Carried Electrodes

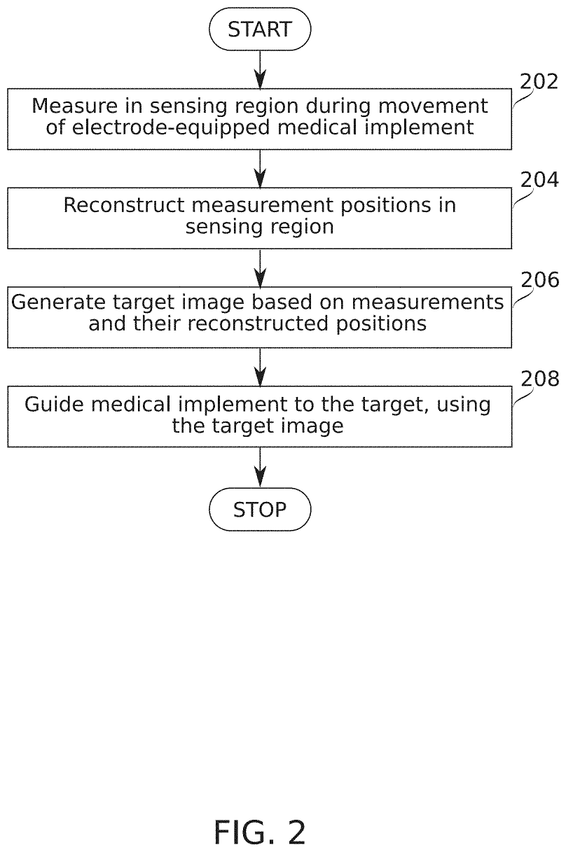

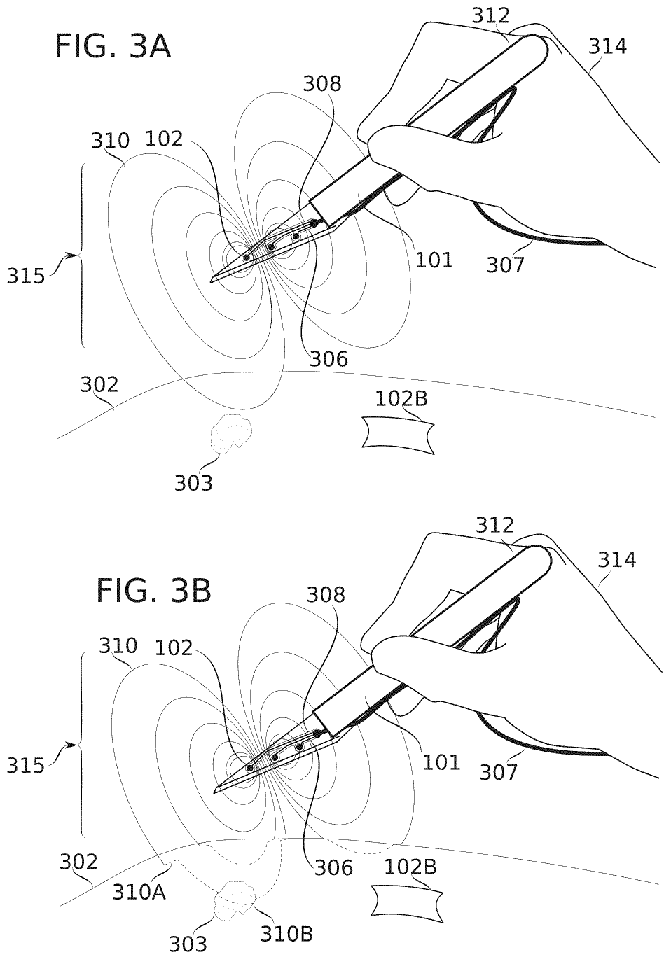

Reference is now made to FIG. 2, which is a flowchart of a method of electrical measurements-based imaging of a body structure, according to some embodiments of the present disclosure. Reference is also now made to FIGS. 3A-3B, which schematically illustrate a medical implement 101 (a scalpel) approaching tissue 302 including a target feature 303 for imaging (which may represent, for example a tumor), according to some embodiments of the present disclosure.

The flowchart begins, and at block 202, in some embodiments, medical implement 101 configured with at least one (e.g. two or more) electrode 102 is moved within a sensing region 315 of a larger region of space which also contains target feature 303 to be imaged. This sensing region 315 may be outside, inside, or partially inside and partially outside a region of tissue (e.g., tissue 302) being imaged. Tissue 302 optionally comprises solid structure, liquid structure, and/or gaseous space, in any combination. The movement is optionally while the medical implement 101 (e.g., a handle 312 of the medical implement 101) is held within the grip of a doctor's hand 314. Optionally, the tool 101 is moved while within the grip of a mechanical manipulator; e.g., a surgical robot.

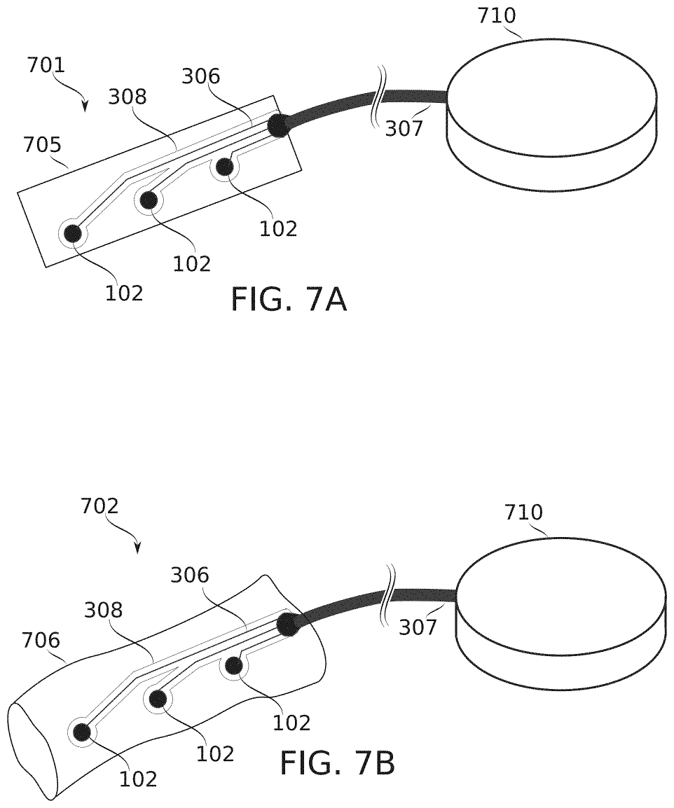

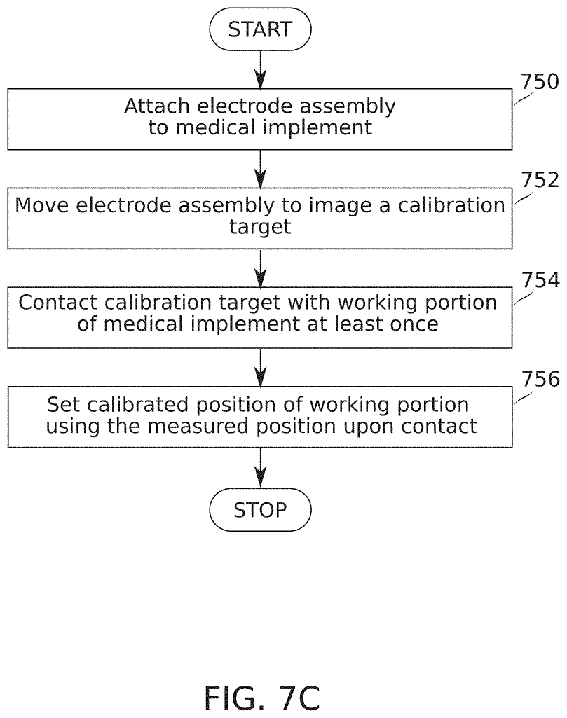

In some embodiments, the electrodes 102 are configured (e.g., by connection via conductive trace 306 and/or connecting cable 307 to a controller 106) to transmit time-varying electrical fields 310 between them (for example one electrode transmitting and one receiving, each electrode transmitting and each electrode receiving all the transmissions, etc.). There may be one or more pairs of electrodes 102. Optionally, electrodes 102 participate in one or more electrode pairs. For example, four electrodes 102 may make up to six pairs. Electrical fields 310 may be transmitted/received between a plurality of groups (e.g., pairs) of electrodes 102, optionally at a plurality of different frequencies. Optionally, electrodes 102 are ganged together in groups larger than a pair, optionally defined by transmission/receiving frequency. For example, one electrode transmits and two receive, two transmit and one receives, or any other suitable grouping. Herein, descriptions of electrical transmission/receiving with respect to a pair of electrodes 102 should be understood as optionally applying to groups of electrodes 102 larger than a pair. In some embodiments, electrodes 102 and/or conductive traces 306 are insulated from the medical implement 101 by insulating material 308. In some embodiments, insulating material 308 is provided as a backing, for example as shown in the embodiments of FIGS. 7A-7B, herein. Optionally, additional electrodes 102B are also used in transmitting and/or receiving electrical fields in a configuration that generates changing electrical field measurements as a function of the movements of electrodes 102 and the tool 101 which carries them.

In a configuration where electrodes 102 are electrical field transmitters: as the electrodes 102 move around in the sensing region 315 (in conjunction with movements of tool 101), so does the electrical field 310 which is transmitted from them. At different positions, electrical currents set up by the electrical field 310 pass though different tissue portions within the environment near the sensing region 315. Insofar as the environment is generally dielectrically inhomogeneous, the electrical parameters (e.g., voltage difference, current, and/or impedance) measured between electrodes 102, and/or electrodes 102 and electrodes 102B change during the movements referred to in block 202. In some embodiments, electrodes 102 are used in a receiving mode while electrodes 102B transmit. Then, although the configuration of electrical field 310 remains static, measurements 110A in sensing region 315 are potentially still influenced by the dielectric structure of tissue 302, so that information can be extracted from the measurements 110A to form an image.

In some embodiments (e.g., in a self-sensing configuration), as a pair of electrodes 102 approaches any particular portion of tissue (for example, advances toward target feature 303 between the position of FIG. 3A and the position of FIG. 3B), that portion of tissue imposes a greater and greater influence on the dielectric parameter(s) which the electrodes 102 measure. For example, comparing FIGS. 3A-3B, isopotential lines of electrical field 310 are shown distorted where they interact with tissue 302 and/or target feature 303, for example, at label locations 310A, 310B.

If the portion of tissue is dielectrically distinct from other parts of the environment which the electrode pair is already in (for example, target feature 303 is dielectrically distinct from the tissue 302), then approaching it, correspondingly, will tend to result in a greater and greater change in measured electrical parameters towards being like the impedance of the target region (i.e., other thing being equal). This change becomes the basis of image production in the remainder of the method according to the presently discussed embodiments.

At block 204, in some embodiments, measurements 110A in the sensing region 315 are reconstructed to a reconstruction 110B of their positions. Optionally, the reconstruction is based on values in the measurements themselves. In some embodiments, the reconstruction procedure, in some embodiments, uses a combination of multidimensional scaling and local coherence constraints, for example as described in U.S. Provisional Patent Application No. 62/546,775 entitled Field Gradient-Based Remote Imaging, and/or in International Patent Application No. PCT IB2018/050192 entitled Systems and Methods for Reconstruction of Intra-body Electrical Readings to Anatomical Structure, the contents of each of which are included by reference herein in their entirety. In overview, the result of this reconstruction is a 3-D model of sensing region 315. In some embodiments, the model also models surroundings of sensing region 315.

In some embodiments, reconstruction is based on position determinations made by position monitoring system 107 while the measurements 110A were being made (e.g., each measurement is associated to a particular position of tool 101 that was sensed at the time the measurement was made).

At block 206, in some embodiments, an image 110C of tissue 302 including target feature 303 is constructed, using the 3-D model reconstructed in block 204. In some embodiments, the inverse method is used to estimate the positions of dielectric materials in the region of the tissue 302 which could explain the measurement observations in the sensing region 315. Insofar the dielectric properties of tissues of different compositions are generally known, this in turn may be used to estimate composition of tissue in the region of tissue 302—that is, which tissues may be present that account for the measurement observations.

At block 208, in some embodiments, the medical implement 101 is guided to move to the target, using the image 110C. This step is provided as an example of a particular use of an image 110C, but it is to be understood that in some embodiments, step 208 is omitted.

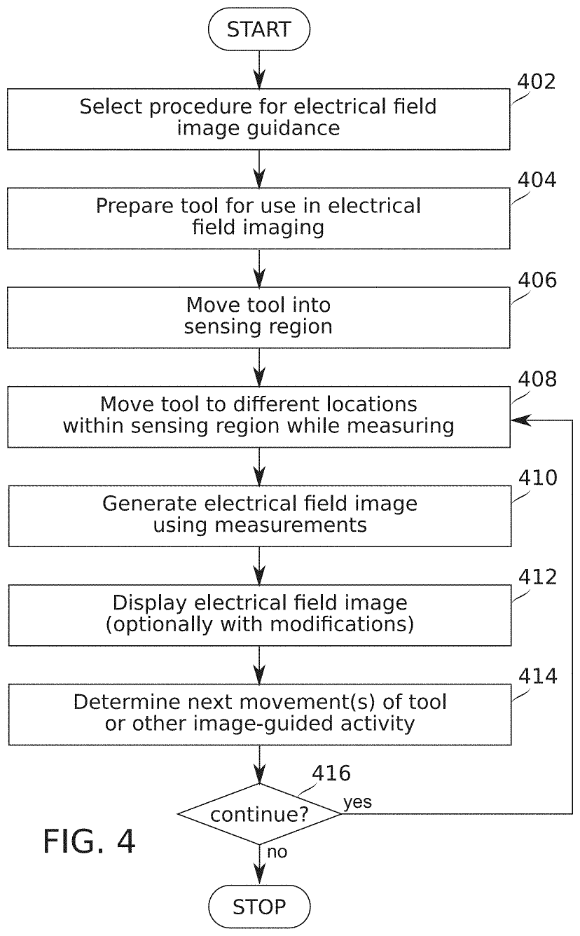

Reference is now made to FIG. 4, which is a flowchart describing creation and use of an electrical field image 110C as part of an overall procedure, according to some embodiments of the present disclosure.

At block 402, in some embodiments, a procedure to be performed using a medical implement 101 is prepared for. This preparation may include selection of the medical implement 101 itself.

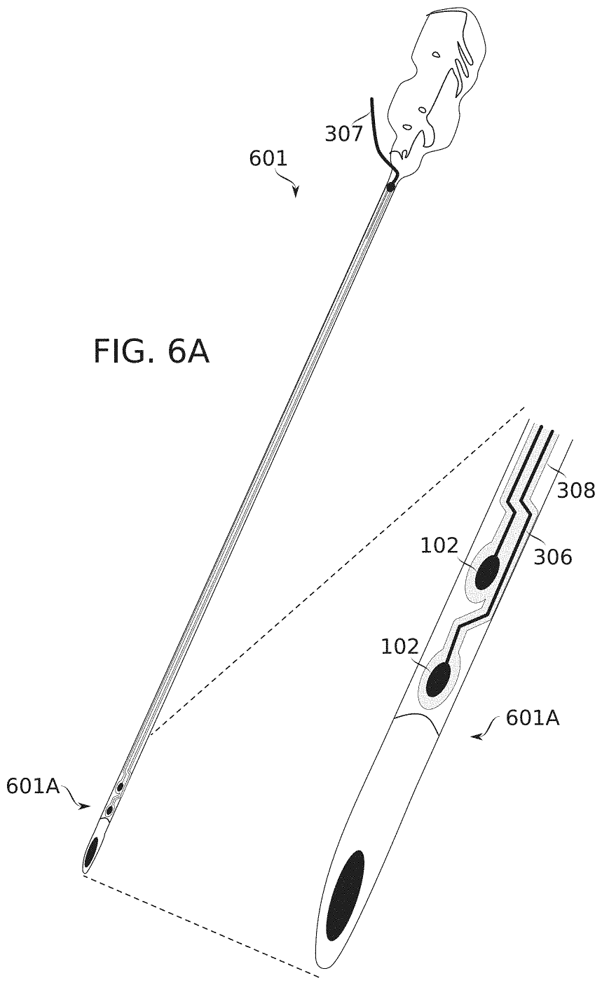

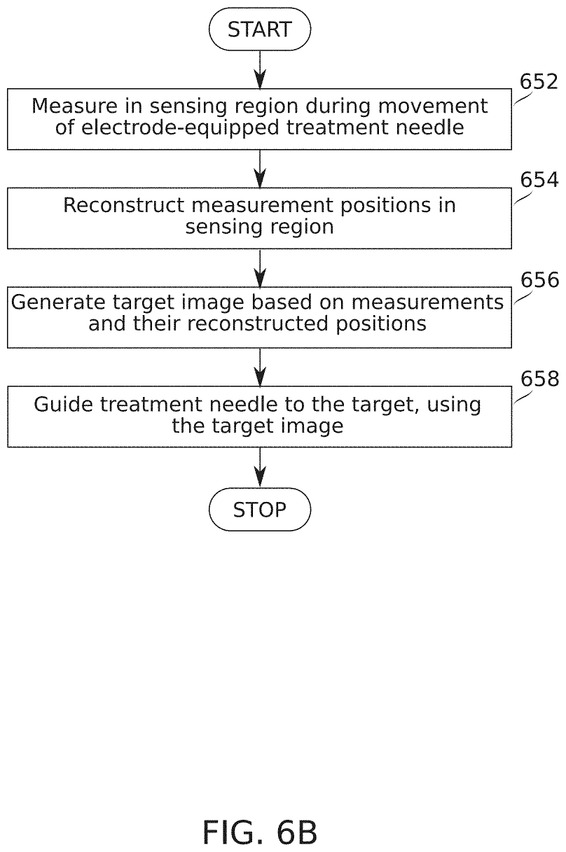

At block 404, in some embodiments, medical implement 101 is prepared for the procedure. In some embodiments, preparation includes attaching one or more electrodes 102 to the medical implement 101; for example, using one of the attachment methods and/or devices described in relation to FIGS. 7A-7B, herein. In some embodiments, medical implement 101 is provided with permanently mounted electrodes; e.g., electrodes printed on the medical implement 101 (for example as described in relation to FIG. 6A), or otherwise mounted. In some embodiments, preparation includes configuring electrodes 102 to be in wired and/or wireless communication with a controller 106. The electrodes are used; for example, in some embodiments (self-sensing embodiments), electrodes 102 are used by controller 106 to transmit at least one time-varying electrical field 310; and also used to act as a receiver for electrical field 310. In some embodiments, electrodes 102 are used to generate electrical field 310 while additional electrodes 102B sense changes due to movements of electrodes 102. In some embodiments, electrodes 102 are used to sense electrical field 310 produced by fixed electrodes 102B. Controller 106 is configured so that it can begin receiving input from electrodes 102 to be converted into measurements of electrical field 310.

At block 406, in some embodiments, electrode-equipped medical implement 101 is brought to a sensing region 315 in the vicinity of tissue 302 so that the at least one time-varying electrical field 310 passes into tissue 302, and/or so that an electrical field 310 generated from some at least one additional electrode 102B and which passes into tissue 302 can be received by electrodes 102. Sensing region 315 optionally comprises regions separated from tissue 302 by internal and/or external fluid (air and/or liquid), and/or other tissue. Additionally or alternatively, sensing region 315 comprises regions in contact with a surface and/or internal portion of tissue 302.

At block 408, in some embodiments, medical implement 101 is moved around within a sensing region 315 while measurements 110A of electrical field 310 are recorded (via one or both of electrodes 102 and additional electrodes 102B); for example, as described in relation to block 202 of FIG. 2. Sensing region 315, in some embodiments, is (at least for production of a first image) a region spatially separate and distinct from a region of tissue 302 to be imaged. Optionally, the extent of sensing region 315 is changed (e.g. increased) during a procedure, for example as medical implement 101 moves and receives signals from in additional locations, for example, as the medical implement is brought closer to tissue 302 and/or some target feature 303 within tissue 302 which is a target for imaging.

At block 410, in some embodiments, an electrical field image 110C comprising tissue 302 and/or target feature 303 is constructed, for example, as described in relation to blocks 204 and 206 of FIG. 2, herein.

At block 412, in some embodiments, electrical field image 110C is displayed. In some embodiments, image 110C is displayed using just information developed from measurements 110A (e.g., via reconstruction of the measurement positions 110B and production of an image therefrom). Optionally, electrical field image 110C is combined with other information for display; for example superimposed on or otherwise shown in relation to an anatomical image obtained from another source such as an MRI image, CT image, image atlas, and/or another source. Optionally, an indication of the position of tool 101 relative to features shown in electrical field image 110C is also displayed together with electrical field image 110C; for example, an image representation of tool 101. In some embodiments, position of the tool is determined with reference to a calibration of the relative positions of electrodes 102 and tool 101 and a portion thereof (for example, working portion 101A).

At block 414, in some embodiments of the invention, electrical field image 110C (along with whatever additional information may be displayed along with it) is used in the determination of a next step of an overall procedure. In some embodiments, the determination comprises identifying a target and/or direction of a target (e.g., target feature 303), and determining to move tool 101 in the direction of that target. In some embodiments, the determination comprises determining to acquire more measurements before identifying and/or moving toward a target. The determination may also be to use tool 101 (e.g., to ablate, inject, and/or gather a sample), and/or to terminate the procedure (for example, after its completion).

Optionally, a determination of a next movement is interrelated with use of a linked tool 104. In some embodiments, the physical position of tool 101 is used as a guide for the positioning of linked tool 104 (e.g., by bringing the two tools together, by passing tool 104 through a guiding portion of tool 101, and/or by placing tool 104 at a particular distance and/or direction from tool 101). In some embodiments, a frame of reference giving the position of linked tool 104 is registered to a frame of reference of electrical field image 110C; for example, by use of a fiducial mark visible in electrical field image 110C, by registration of electrical field image 110C with an anatomical representation that tool 104 can in turn be registered to, or another method.

In some embodiments, linked tool 104 itself is visible in the electrical field image 110C (e.g., since it may itself have effects on the dielectric environment interacting with electrical fields generated from within sensing region 115. In some embodiments, as tool 104 is moved around, new measurements from tool 101 (made while moving tool 101) are also obtained and constructed into a new and/or updated electrical field image 110C.

In some embodiments, linked tool 104 may have an operational mode (e.g., a mode which itself emits and/or absorbs electrical energy) which could potentially interfere with imaging using medical implement 101. In some embodiments, imaging by tool 101 and use of tool 104 are gated to each other (one-way or two-way gating) so that measurements which could potentially be affected by interference are not made, or treated differently (e.g., discarded, differentially processed, and/or segregated into data used to produce a different image).

At block 416, in some embodiments, a decision is made as to whether to terminate imaging within the procedure, based on the determination of block 414. If imaging is to continue, the procedure continues with a return to block 408. Otherwise, the procedure ends.

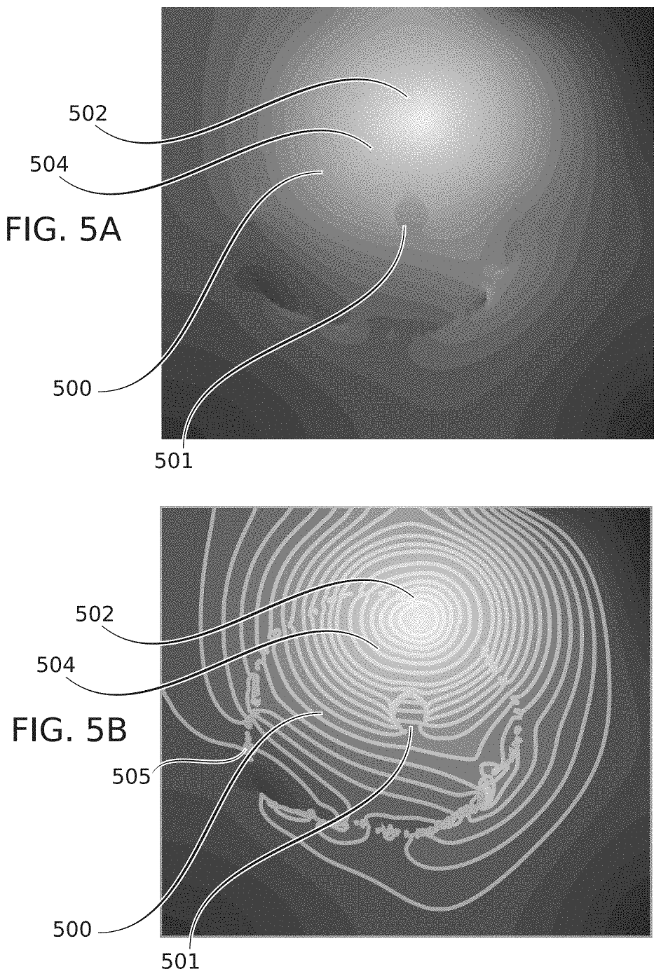

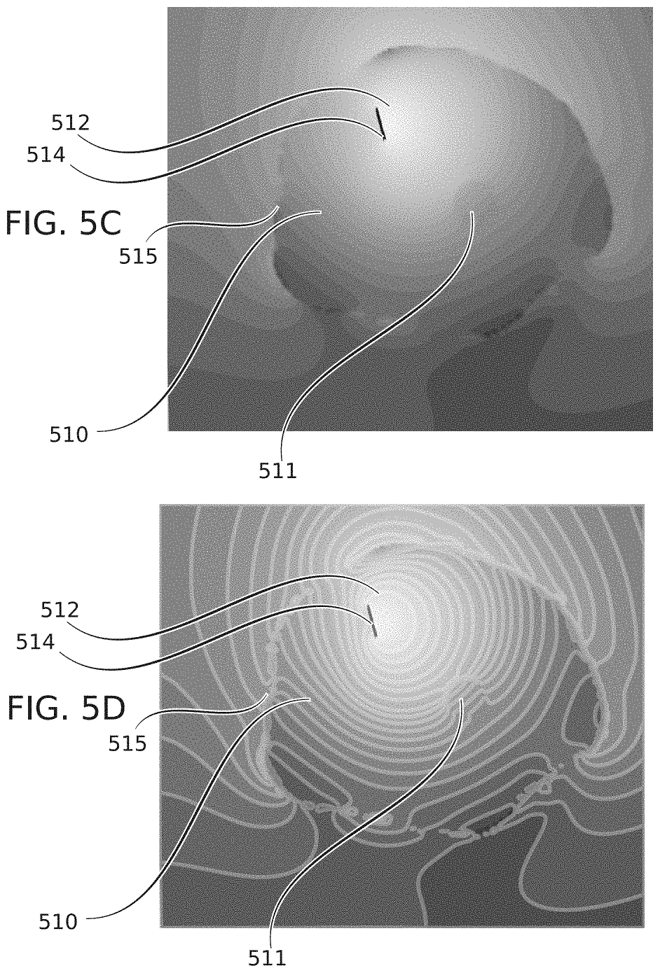

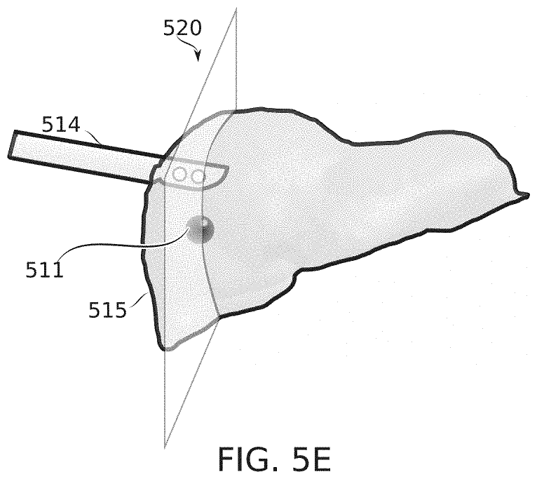

Reference is also made to FIGS. 5A-5D, which show cross-sections of a liver image 510 produced by simulation of electrical field measurements made during simulated movements of an electrode-equipped scalpel 514 moving within an electrical field while measuring that electrical field, according to some embodiments of the present disclosure. Further reference is made to FIG. 5E, which shows relative positions of a scalpel 514 and a liver 515, and the plane 520 used in the simulation, results of which are shown in the images of FIGS. 5A-5D, according to some embodiments of the present disclosure.

### Images Produced by Electrical Field Imaging Using Medical Implement-Carried Electrodes

FIGS. 5A-5D are images produced from electrical measurement information simulating the measurements that would be made by a pair of receiving electrodes fixedly mounted on a medical instrument 514 such as a scalpel, while the scalpel moves within region 512. The measurements are used to create an image of a simulated liver 515 bearing a simulated tumor 511, each having distinct dielectric properties. Electrical field transmitting electrodes (corresponding, e.g., to electrodes 102B of FIG. 1) were simulated as being placed on a surface of the liver.

The image contents of FIGS. 5A and 5B are identical, except that gradient lines have been superimposed on FIG. 5B to help emphasize certain structural details. The image contents of FIGS. 5C and 5D are also identical, except that gradient lines have been superimposed on FIG. 5D to help emphasize certain structural details.

In the case of FIGS. 5A-5D, electrode pairs operate with one electrode transmitting, and the other receiving. The frequency of transmission used was 12.8 kHz. The tumor simulated is a sphere 20 mm in diameter, and located at a depth 45 mm within the liver.

In some embodiments, transmitting electrodes are provided at a fixed position, e.g., placed on a surface such as a liver surface as is simulated in the case of FIGS. 5A-5D. As the medical instrument 514 moves, the electric field characteristic(s) that its electrodes measure changes as a function of position. In particular, the distribution of measurements in space is affected by the dielectric properties of tissues through which currents of the electric field travel, even at positions away from the tissue itself (e.g., outside an exterior surface of the tissue). For example, tumor 501, 511 has different dielectric properties than tissue of the surrounding liver 500, 515. As more positions within region 502, 512 are sampled, a three-dimensional set of measurements is gradually built up. This is reconstructed into a three-dimensional model of the electrical field, and finally an image of the tissue itself is calculated from the electrical field model, by calculating what arrangement of dielectric materials existing in the direction of the tissue would account for the particular pattern of measurements (e.g., gradients in that pattern of measurements) seen in the three-dimensional model.

In some embodiments, an additional or alternative transmitting/receiving arrangement is optionally provided. In some embodiments, the moving electrodes are transmitting, and one or more fixed electrodes are receiving. Here, the overall electrical field itself is what changes “moving through” positions of the receiving electrodes.

Additionally or alternatively, electrodes affixed to medical instrument 514 both transmit and receive. As the medical instrument 514 or catheter moves, the electric field it transmits through its surroundings changes as a function of differences in dielectric properties tissues through which currents of the field travel. The remote electrical field changes are in turn sensed back at the electrodes of medical instrument 514, e.g., as one or more of a change in current and/or voltage. In this case, the accumulated measurements, rather than revealing voltage gradients of some particular electrical field, reveal gradients of change in measurements, as a function of position, of an electrical field which is also dynamic as a function of position. Again, the measurements at each position are different from neighboring measurements because of different influences on electric field transmission by nearby structures, such as liver 500, 515 and tumor 502, 512.

In some embodiments, in order to assign specific positions to measurements (and thereby obtain a structure from which spatial gradients of those measurements can be obtained), reconstruction is performed using the measurements taken. In some embodiments, this reconstruction uses an approach which combines multi-dimensional scaling (MDS) and local spatial coherence. This method has the particular potential advantage of making the measuring device “self-tracking” based on the measurements themselves. Additionally or alternatively, another method is used to track where the electrodes are at the time of each measurement. For example, an optical device is optionally set up to image the three-dimensional position of the medical instrument 514, and/or a robotic device is used to manipulate medial instrument 514, with the position of the robotic device being recorded during movements accompanying measurement.

Finally, reconstruction of the images shown in FIGS. 5A-5B and 5C-5D performed by attributing features of the spatial distribution of measurement gradients in regions 502, 512 to differences in dielectric properties in remotely located structures, for example by using the inverse method. Optionally, the inverse method is guided by constraints such as a known anatomical shape of nearby features (like the liver) obtained from previous imaging, anatomical atlas data, and/or known dielectric properties of nearby features.

### Electrical Field Imaging Using an Electrode-Carrying Needle

Reference is now made to FIG. 6A, which schematically illustrates a needle 602 (e.g., a biopsy needle and/or an ablation needle) for use in a medical procedure, and provided with two electrodes 102, according to some embodiments of the present disclosure.

Region 601A of needle 601 is repeated in the magnified inset view to show details including electrodes 102, conductive traces 306, and insulating material 308. In some embodiments of the invention, one or more of elements 102, 306, 308 are printed onto the needle, for example using inkjet printing which alternates an electrically non-conductive material (e.g., for insulating material 308) with an electrically conductive material (e.g., for electrodes 102 and conductive traces 306). In some embodiments, an assembly of electrodes is provided to needle 601 using a sleeve (e.g., an elastically or otherwise fitted sleeve) or adhesive attachment, for example as described in relation to FIGS. 7A-7B. In some embodiments, connecting cable 307 is used to interconnect between conductive traces 306 and controller 106, optionally via a wireless transmitter 710, for example as described in relation to FIGS. 7A-7B.

Reference is now made to FIG. 6B, which is a flowchart describing a procedure for use of needle 601 in a medical procedure, according to some embodiments of the present disclosure. Needle 601 is optionally a biopsy needle and/or an ablation needle. The procedure of FIG. 6B refers to either type of needle as a “treatment needle”. Certain referenced features (e.g., tissue-related features) not shown in FIGS. 6A-6B may be found, for example, in FIGS. 3A-3B.

It should be understood that while the method of FIG. 6B is described in relation to a treatment needle, these descriptions also serve as a non-limiting example of how the general method of FIG. 2 is optionally applied to more particular medical implements 101 having a designated medical use, for example a scissors, scalpel, knife, cannula, clamp, needle, laparoscope, or another tool 101 having a designated medical use, for example as described in relation to FIG. 1.

The flowchart begins, and at block 652, in some embodiments, treatment needle 601 configured with a plurality of electrodes 102 is moved within a sensing region 315 of a larger region of space which also contains target feature 303 to be imaged. The sensing region may be outside, inside, or partially inside and partially outside a region of solid tissue (e.g., tissue 302).

In some embodiments of the invention, target feature 303 comprises one or more liver nodules. Liver nodules comprise growths of liver cells (hepatocytes) which potentially associated with risk and/or harm by their size, position (e.g., blocking or threatening to block blood flow in a hepatic blood vessel), and/or risk of metastasizing. Optionally, needle 601 is a biopsy needle, to be guided to a liver nodule to sample it for testing. Optionally, needle 601 is an ablation needle, to be guided to a liver nodule to administer a treatment (such an injected substance, delivery of energy, or another treatment) targeted to control risk and/or harm due to the nodule; e.g., by ablating it.

The electrodes 102 are configured (e.g., by connection via conductive trace 306 and/or connecting cable 307 to a controller 106) to transmit and/or receive time-varying electrical fields 310, for example as described in relation to block 202 of FIG. 2.

As a pair of electrodes 102 moves around in the sensing region 315 (in conjunction with movements of treatment needle 601), so does the electrical field 310 which is transmitted between them. At different positions, electrical currents set up by the electrical field 310 pass though different tissue portions within the environment near the sensing region 315. Insofar as the environment is generally dielectrically inhomogeneous, the impedance measured between electrodes in electrode pairs changes during the movements of block 652.

For example, as a pair of electrodes approaches any particular portion of tissue (for example, advances toward target feature 303 between the position of FIG. 3A and the position of FIG. 3B), that portion of tissue imposes a greater and greater influence on the electrical parameters which the electrodes measure. For example, comparing FIGS. 3A-3B, isopotential lines of electrical field 310 are shown distorted where they interact with tissue 302 and/or target feature 303, for example, at label locations 310A, 310B.

If the portion of tissue happens to be dielectrically distinct from the environment which the electrode pair is already in (for example, target feature 303 is dielectrically distinct from the tissue 302), then approaching it, correspondingly, will tend to result in a greater and greater change in measured electrical parameters towards being like the electrical parameters of the target region (i.e., other thing being equal). This change may become the basis of image production in the remainder of the method.

At block 654, in some embodiments, measurements 110A in the sensing region 315 are reconstructed to a reconstruction 110B of their positions, based on values in the measurements themselves. In some embodiments, the reconstruction procedure uses a combination of multidimensional scaling and local coherence constraints, for example as described in U.S. Provisional Patent Application No. 62/546,775 entitled Field Gradient-Based Remote Imaging, and/or in International Patent Application No. PCT IB2018/050192 entitled Systems and Methods for Reconstruction of Intra-body Electrical Readings to Anatomical Structure, the contents of each of which are included by reference herein in their entirety. In overview, the result of this reconstruction is a 3-D model of sensing region 315, which may also model surroundings of the sensing region.

At block 656, in some embodiments, an image 110C of tissue 302 or a 3-D model thereof including target feature 303 is constructed, using the measurements reconstruction 310B of block 654. In some embodiments, the inverse method is used to infer the positions of dielectric materials in the region of the tissue 302 which could explain the measurement observations in the sensing region 315.

At block 658, in some embodiments, the treatment needle 601 is guided to the target, using the image 110C. This step is provided as an example of a particular use of an image 110C, but it is to be understood that the method of FIG. 2 is performed just up to block 656, in some embodiments.

### Add-On Electrode Assemblies

Reference is now made to FIG. 7A, which schematically illustrates an adhesive electrode assembly 701, for use by attachment to a medical implement, according to some embodiments of the present disclosure. Such a use is referred to herein as an “add-on” use. Reference is also made to FIG. 7B, which schematically illustrates a sleeve-attached electrode assembly 702, for use by attachment to a medical implement, according to some embodiments of the present disclosure.

Each of FIGS. 7A, 7B show electrodes 102, electrically insulating material 308, conductive traces 306, connecting cable 307, and wireless transmitter 710.

Wireless transmitter 710 provides an optional example of how portions of controller 106 related to electrical field generation and received electrical field signal communication may be implemented, in some embodiments. In some embodiments, wireless transmitter 710 comprises circuitry configured to transmit electrical field measurements (signals) received from electrodes 102 to a central portion of controller 106 that carries out further processing. Optionally, wireless transmitter 710 is packaged together with circuitry configured to receive and digitize electrical field measurements received from electrodes 102 before transmission.

In some embodiments, wireless transmitter 710 is interfaced with (and optionally packaged with) an electrical field (signal) generator, configured to generate electrical fields (e.g., radio frequency electrical fields) transmitted through electrodes 102. Control of the electrical field generator from other portions of controller 106 is optionally exercised through wireless transmitter 710.

In some embodiments, adhesive electrode assembly 701 comprises an adhesive-backed surface 705 which can be adhered to a receiving surface of medical implement 101. Surface 705 optionally comprises a flexible substrate (e.g., a silicon polymer) and/or a rigid substrate (e.g., a metal or plastic card).

In some embodiments, sleeve-attached electrode assembly 701 comprises a sleeve 706 which can be fit over to a receiving surface of medical implement 101. Optionally sleeve 706 comprises an elastic material which is stretched to fit elastically over a portion of medical implement 101; for example, stretched to fit over a tube and/or handle of medical implement 101.

Reference is now made to FIG. 7C, which is a flowchart describing a method of attaching electrodes 102 to a medical implement and calibrating the resulting implement for use in electrical field imaging, according to some embodiments of the present disclosure.

The flowchart begins, and at block 750, in some embodiments, an electrode assembly comprising electrodes 102 (e.g., electrode assembly 701, 702, or another electrode assembly) is attached to a medical implement 101.

At block 752, in some embodiments the electrode assembly is moved (by movement of medical implement 101) to image a calibration target. The imaging may be, for example, as described in relation to blocks 202, 204, and 206 of FIG. 2. The calibration target, in some embodiments, is a phantom configured to dielectrically induce effects like those of living tissue on an electrical field. The imaging is optionally by use of an electrical field both transmitted from and received by electrodes 102, transmitted from electrodes 102 and measured by additional electrodes 102B, and/or transmitted from additional electrodes 102B and received by electrodes 102.

The relative position of the electrodes 102 during the imaging is tracked, for example using a method that directly relies on use of the electrical field measurements, and/or using another method (e.g. implemented by position monitoring system 107).

At block 754, in some embodiments a portion (for purposes of describing this flowchart, working portion 101A; optionally another portion) of medical implement 101 is brought into contact with a known portion (e.g., a surface or interior portion) of the calibration target. Optionally, the known portion is marked or otherwise preselected. Optionally, the known portion is determined by another method, for example, video monitoring of the calibration target, and/or sensing (e.g., capacitance-based tracking, for example as used in trackpad computer input devices) by the calibration target itself.

This operation is optionally performed a plurality of times, and optionally to a plurality of contact positions and/or a plurality of angles of contact. During contact, the position of the electrodes 102 is also recorded (by whatever positioning method is used, for example based on reconstruction of a space using the electrode measurement values themselves, and/or using an external position monitoring system 107. Positions of the electrodes 102 are thereby corresponded with positions of working position 101A in contact with the calibration target.

At block 756, in some embodiments, a calibration function for determining a calibrated position of the working portion 101A with respect to the position of electrodes 102 is determined, using the corresponded positions. The calibration function is optionally a 3-D function that allows transforming a measured electrode position into a calibrated position of working portion 101A, or whatever other portion of medical implement 101 was calibrated to. Optionally, calibration of the position of working portion 101A (and optionally any portion of medical implement 101) is indirectly determined by calibration of a different portion of medical implement 101, for example using known relative positions of portions on the medical implement 101.

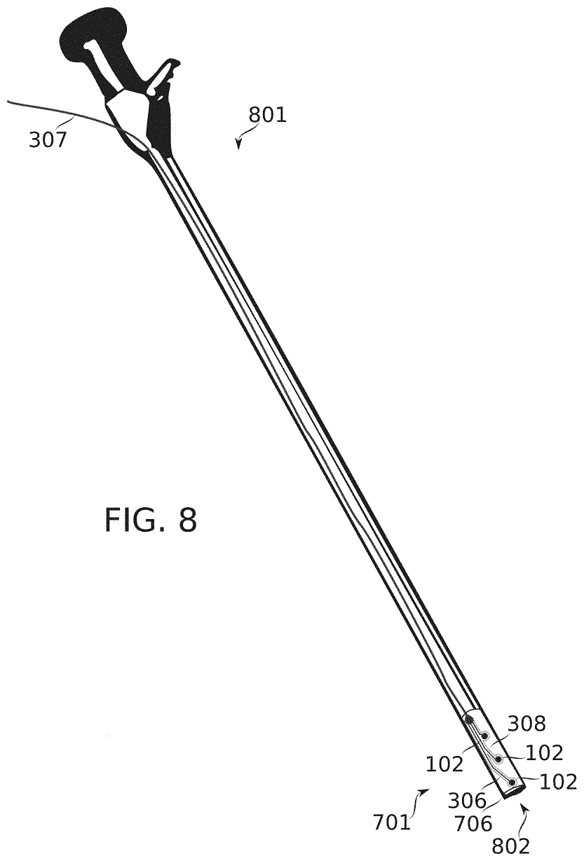

Reference is now made to FIG. 8, which schematically illustrates a laparoscope 801 provided with a plurality of electrodes 102, according to some embodiments of the present disclosure.

In some embodiments, electrodes 102 are secured to a portion of laparoscope 801 using an attachable electrode assembly such as sleeve-attached electrode assembly 701 (including, in some embodiments, sleeve 706, electrodes 102, conductive traces 306, connecting cable 307, and/or insulating material 306). The working portion 101A of laparoscope 801 is optionally assigned to be any suitable portion of the laparoscope 801, for example, laparoscope distal end 802.

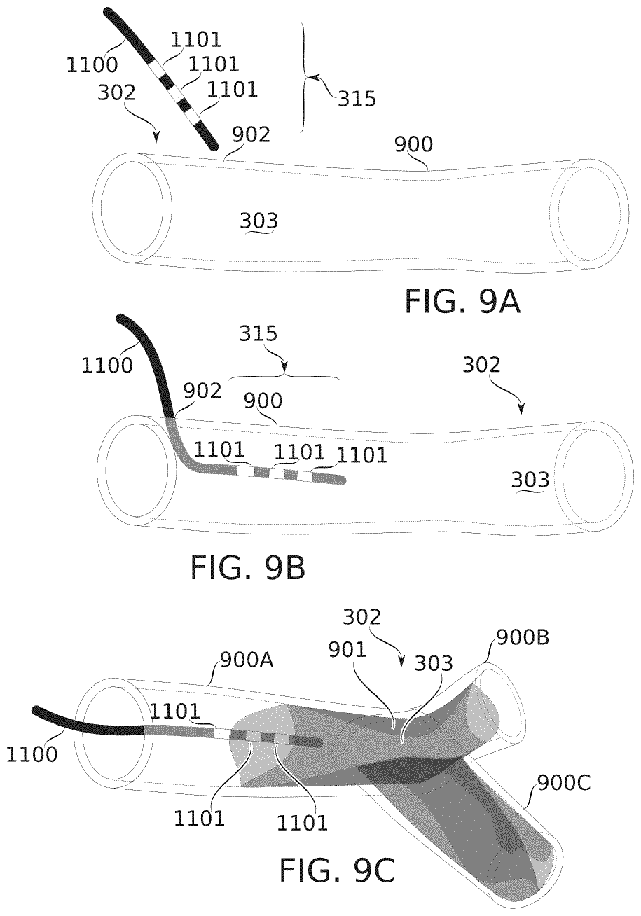

Reference is now made to FIG. 11, which schematically illustrates a guidewire 1100 (e.g., a catheter guidewire) equipped with electrodes 1101, according to some embodiments of the present disclosure. In some embodiments, the guidewire is used for performing of coronary studies and/or procedures, and is optionally used to provide electrical imaging at a coronary target, and/or at one or more stages along the way to reaching a target, for example as described in relation to FIGS. 9A-9C.

In some embodiments, guidewire 1100 comprises a long (_e.g. 220 cm, optionally 80-240 cm), thin (e.g._, about 0.032″ diameter) member, comprising one or more distally located clusters 1104, 1106 of electrodes 1101. In some embodiments, the electrode length is about 0.5 mm.

Electrode cluster 1104 comprises a group of electrodes 1101 (optionally three electrodes, or another number of electrodes) positioned along a flexible, remotely steerable tip 1102 of guidewire 1100. In some embodiments, an inter-electrode spacing between tip electrodes is about 1 mm.

In some embodiments, steerable tip 1102 is configured to be bendable, for example, through a range of curvature between straight and a 3 mm radius curvature.

Electrode cluster 1106 comprises a group of electrodes 1101 (optionally four electrodes, or another number of electrodes) positioned along a shaft region of guidewire 1100 proximal to the steerable tip 1102. In some embodiments, an inter-electrode spacing between tip electrodes is about 2 mm.

In some embodiments, electrodes 1101 are formed from electrode wires, each separately coated with an insulating material, and exposed at the positions shown. Optionally, the electrode wires serve as part of a wire braiding and/or coil structure which makes up the main length of the guidewire 1100. Additionally or alternatively, the electrode wires extend longitudinally via a central lumen of guidewire 1100.

### Electrical Field Imaging Use Cases—Guidewire and Imaging Electrodes

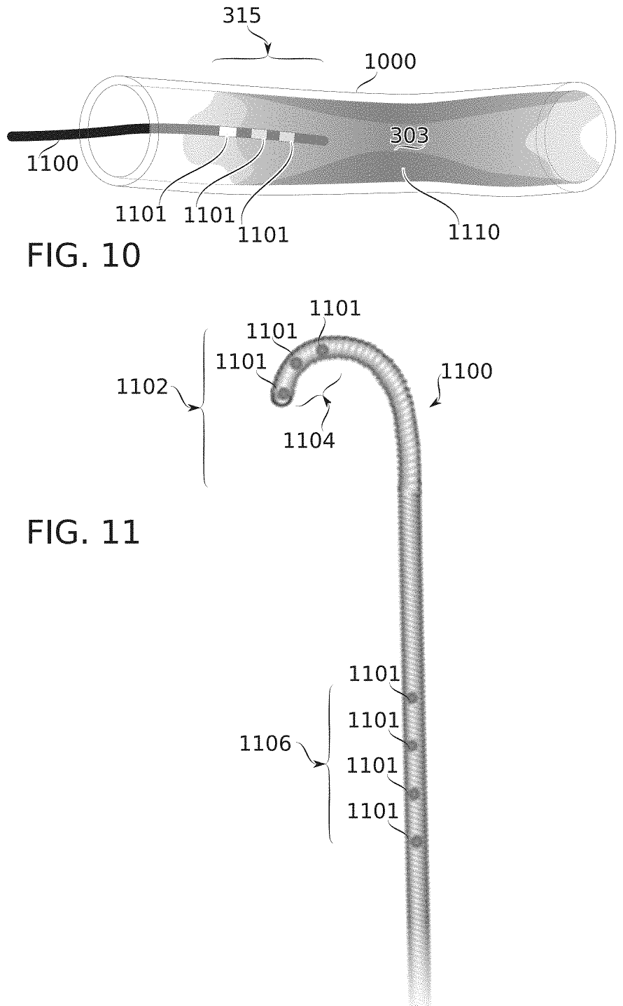

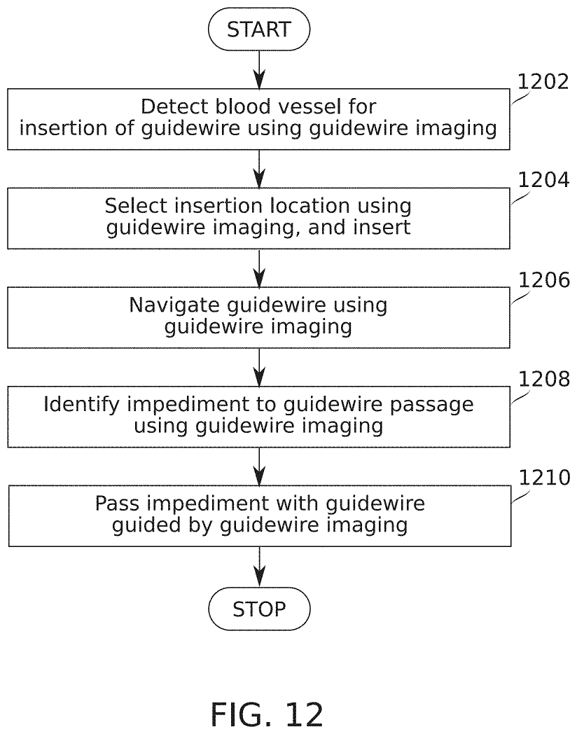

Reference is now made to FIGS. 9A-9C, which schematically illustrate stages in the insertion to a body of an electrode-equipped guide-wire 1100 configured for electrical imaging, according to some embodiments of the present disclosure. Reference is also made to FIG. 10, which schematically illustrates an electrode-equipped guidewire 1100 configured for electrical imaging, shown in relation to a stenotic blood vessel 1000. Further reference is now made to FIG. 12, which is a flowchart describing use of a guidewire 1100 for imaging, according to some embodiments of the present disclosure.

A self-imaging guide wire provides a potential advantage for use in a microcatheter procedure, by allowing the device itself to detect its surroundings, optionally in sufficient detail to support decision making at one or more key junctures of the procedure. In some embodiments, the guidewire image is potentially of sufficient quality as to obviate a need to activate another imaging modality, such as an X-ray imaging modality. Optionally, equipment for performing X-ray imaging is omitted from an operating room in which a catheterization procedure is being performed. It should be understood that in some embodiments, the electrical field imaging is performed (additionally or alternatively to using electrodes on a guidewire) using electrodes carried on another component used in catheterization, for example, a microcatheter component which is advanced over the guidewire

At block 1202, in some embodiments, a blood vessel insertion point for a guidewire is detected by imaging using electrodes 1101 of guidewire 1100, for example as illustrated in FIG. 9A.

In FIG. 9A, guidewire 1100 (bearing electrodes 1101) is about to be inserted into a large blood vessel 900, for example a femoral artery. In some embodiments, this represents an example of the relative positions of a sensing region 315, tissue 302 (outside of blood vessel 900) and a target feature 303 (blood vessel 900 itself). This configuration may be useful, for example, to assist in localization of an insertion point 902 for a guidewire 1101.

At block 1204, in some embodiments, guidewire 1100 is inserted into selected insertion point 902. In FIG. 9B, for example, guidewire 1100 has passed into blood vessel 900, and is imaging ahead of itself (toward target feature 303, from measurements made in region 315) as it is advanced along blood vessel 900. In this case, target feature 303 is a normal-appearing extent of blood vessel.

At block 1206, in some embodiments, guidewire 1100 is advanced through the vascular system, e.g., from large blood vessel 900 to a blood vessel 900A (FIG. 9C). In FIG. 9C, guidewire 1100 is imaging ahead of itself to a region 303 which comprises a partial stenotic block at a vascular junction, from measurements made in region 315) as it is advanced along blood vessel 900.

At block 1208, in some embodiments, an impediment to guidewire passage is identified using guidewire imaging. For example, imaged target feature 303 potentially comprises or both of the branches ahead (branch vessels 900B and 900C), and/or parts of stenotic region 901.

In another example, illustrated in FIG. 10, guidewire 1100, advancing along a blood vessel 1000, is used while electrodes 1101 are moving to perform imaging measurements of electrical fields affected by passage through stenotic imaged region 303.

At block 1210, in some embodiments, the impediment identified at block 1208 is passed, guided by guidewire imaging. For example, in some embodiments, the image produced is optionally used to help determine how to position catheter 1100 (e.g., by manipulation of steerable tip 1102) in order to transit and/or treat stenosis 901, 1110.

### Electrical Field Imaging Use Cases—Needle, Guidewire and EP Catheter

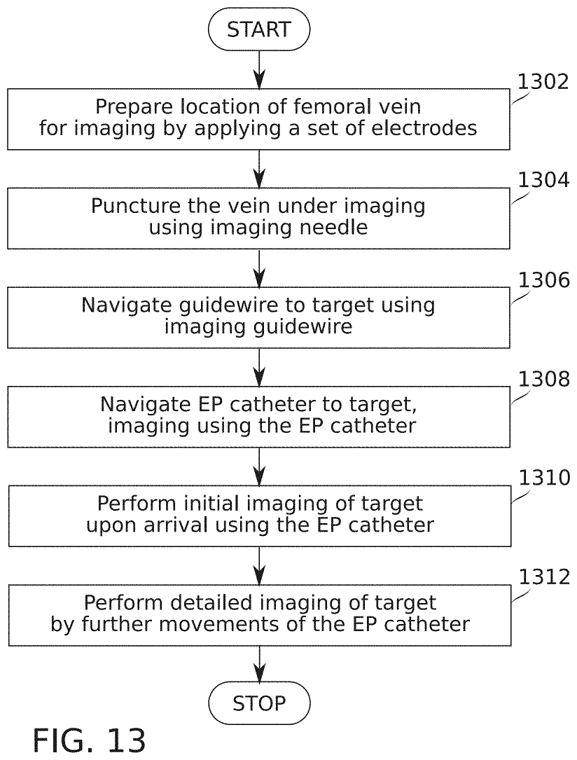

Reference is now made to FIG. 13, which is a flowchart describing use of electrode-based imaging tools during the course of a medical procedure, according to some embodiments of the present disclosure. At an introducing stage of a catheter procedure, an imaging needle or other electrode-bearing device can be used for imaging to assist in introduction of a guidewire to a body, without the use of X-ray radiation and/or contrast agent injection.

At block 1302, in some embodiments, a location of a blood vessel (e.g., a femoral vein) is prepared for imaging by applying (e.g., to the surface of the skin) a set of electrical field-transmitting electrodes configured together with an electrical field generator, so that one or more electromagnetic fields are transmitted through a region which includes the targeted blood vessel. A targeted blood vessel, in some embodiments, comprises an anatomical feature distinct from its surroundings in terms of its effect on the bending of electrical fields in its vicinity, allowing it to become a potential target for electrical field imaging. Larger diameter blood vessels, such as the femoral vein, potentially have larger effects on electrical field bending.

At block 1304, in some embodiments, an electrode-bearing imaging needle (e.g., a needle 601, for example as described in relation to FIG. 6A) is brought into the vicinity of the femoral vein for imaging: that is, brought to a region outside the surface of the skin and within the electrical field which is transmitted from the electrical field-transmitting electrodes. The imaging needle is then moved around outside the body while electrical field measurements 110A are recorded. Positions of the needle during measurement are optionally determined using a position monitoring system 107; for example, using optical tracking, priorly available knowledge (e.g., simulations) about the general structure of the transmitted electrical field, and/or constraints determined by the arrangement of electrodes on the imaging needle such as their distances from each other. The electrical field measurements 110A are converted into a reconstruction 110B of measurements and their positions. This in turn is converted into an image 110C of anatomical features outside the measured region which influence distortions (bending) in the electrical field inside the measured region.

Imaging using the imaging needle continues until the location of the femoral vein is identified. The imaging optionally identifies quality of the blood vessel wall (e.g., not excessively calcified and/or scarred, for example by previous insertions), and/or a shape and/or orientation of the target blood vessel (e.g., a blood vessel region comprising a suitable bend for receiving guidewire 100 at an angle that allows guidewire 100 to be reoriented for proceeding along the blood vessel without reaching a blind end and/or without introduction of force which is potentially injurious).

Then, optionally under image guidance, the imaging needle is inserted to the femoral vein. Alternatively, the penetration is by a non-imaging needle (or by non-imaging introducer).

Optionally, imaging continues during insertion. It should be noted, however, that the needle insertion under image guidance is somewhat different from the earlier imaging; insofar as the needle is introduced, in this stage, into the area being imaged, rather than continuing to image it remotely. For example, the needle position is tracked (e.g., by position monitoring system 107) compared to positions of anatomical features known from the imaging.

Optionally, tracking during insertion uses the image as a position tracking reference: since the image production includes, in some embodiments, estimating about electrical field properties in the imaged region, information about the needle position can potentially be estimated from what it measures electrically as it enters that imaged region.

It may be noted that imaging to find the femoral vein (or another blood vessel) using an imaging needle 601 is optionally a replacement for blood vessel-locating imaging using a guidewire 100, for example as described in FIG. 9A and/or blocks 1202-1024 of FIG. 12.

In some embodiments, use of an imaging needle is omitted, and the procedure begins at block 1306 (e.g., after insertion of a guidewire introducer such as a needle in some other fashion).

At block 1306, in some embodiments, an imaging guidewire 1100 (that is, a guidewire 1100 including electrodes 1101 configured for measuring electrical fields) is inserted to a blood vessel from the entry point discovered using the imaging needle. Optionally, the imaging guidewire 1100 is inserted to the blood vessel through the imaging needle 601 of block 1304. Imaging guidewire 1100 is navigated to a more distal target body cavity of the procedure (e.g., navigated to enter a heart), while making measurements which are used to produce further images. In some embodiments, this corresponds to the operations of block 1206 of FIG. 12 and/or descriptions of FIGS. 9C and/or M. The electrical fields measured are optionally transmitted from body surface electrodes, transmitted from electrodes on another probe inserted to the body, and/or transmitted from electrodes of the guidewire itself.

In some embodiments, use of an imaging guidewire is omitted, and the procedure begins at block 1308 (e.g., after guiding the guidewire in some other fashion to a target).

At block 1308, in some embodiments, an electrophysiology catheter (EP catheter) is navigated to the target body cavity reached by the guidewire. In some embodiments, progress of the EP catheter is monitored by making electrical field measurements from electrodes of the EP catheter, and optionally doing one or more of the following:
Locating the EP catheter by matching EP catheter measurements to measurements obtained during the navigation of the imaging guidewire 1100.
Locating the EP catheter by matching new images made from EP catheter measurements to images obtained during the navigation of the imaging guidewire 1100.
Locating the EP catheter by matching EP catheter measurements to measurements expected in regions imaged during the navigation of the imaging guidewire 1100.
Locating the EP catheter by integrating EP catheter measurements into one or more images obtained during navigation of the imaging guidewire 1000.

Optionally, the measurements by the EP catheter electrodes are used to enhance the images already made during navigation of the imaging guidewire

At block 1310, in some embodiments, the EP catheter reaches the procedure's target body cavity, which may comprise, for example, one or more cardiovascular lumens, such as a heart chamber; for example, a right atrium and/or left atrium. The EP catheter is now used for imaging, for example, using a mapping procedure such as described in International Patent Application No. PCT IB2018/056158 filed Aug. 16, 2018; and entitled “FIELD GRADIENT-BASED REMOTE IMAGING”, the contents of which are included herein by reference in their entirety. Initial imaging, in some embodiments, comprises making measurements by movements of a probe end of the EP catheter bearing the electrodes through a target body cavity to traverse regions near a central region of the target body cavity.

At block 1312, in some embodiments, the EP catheter continues imaging based on movements of the EP catheter which visit regions of the target body cavity in more detail.

### General

It is expected that during the life of a patent maturing from this application many relevant medical implements will be developed; the scope of the term medical implement is intended to include all such new technologies a priori.

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

What is claimed is:

1. A method of imaging using movements of a medical implement comprising a working portion and at least two electrodes affixed to the medical implement, wherein the working portion is configured to manipulate an internal anatomical structure upon reaching a position of mechanical contact therewith, the method comprising:
transmitting an electrical field from at least one transmitting electrode to interact with an internal anatomical structure;
moving the working portion of the medical implement among positions away from the position of mechanical contact, while the transmitted electrical field interacts with the internal anatomical structure;
receiving the electrical field by at least one receiving electrode;
measuring changes in the electrical field due to the moving and the interacting with the internal anatomical structure; and
constructing an image of the internal anatomical structure, based on the measuring;
wherein the at least two electrodes affixed to the medical implement comprise at least one of the at least one transmitting electrode and the at least one receiving electrode.

2. The method of claim 1, wherein the at least two electrodes comprise both the at least one transmitting electrode and the at least one receiving electrode.

3. The method of claim 1, wherein the at least two electrodes comprises an electrode covering a surface of the working portion.

4. The method of claim 1, comprising moving the working portion to the position of mechanical contact, based on the position of the working portion relative to a position of the internal anatomical structure shown in the image.

5. The method of claim 4, comprising displaying the image to a user, along with a representation of the working portion at a current position relative to the internal anatomical structure.

6. The method of claim 1, wherein the constructing comprises estimating a composition of tissue in the region of the anatomical structure, based on the measuring.

7. The method of claim 6, wherein the estimating is based on estimating a distribution of dielectric properties in the region of the anatomical structure which accounts for changes measured.

8. The method of claim 1, wherein the constructing comprises associating particular positions of the at least one electrode to the changes measured in the electrical field due to the moving and the interacting.

9. The method of claim 8, wherein the associating particular positions is based on at least one distance between a plurality of electrodes of the at least one electrode.

10. The method of claim 1, wherein the at least one electrode comprises three or more electrodes, each at a known distance from another of the three or more electrodes.

11. The method of claim 1, wherein the medical implement is held at a handle which controls positioning of the working portion, and the handle and the working portion are rigidly interconnected.

12. The method of claim 1, wherein the medical implement is held at a handle which controls positioning of the working portion, and the handle and the electrodes are rigidly interconnected.

13. The method of claim 11, wherein the medical implement is a scalpel.

14. The method of claim 1, wherein the working portion is moved by manipulation of the medical implement while holding the medical implement within 20 cm of the working portion.

15. The method of claim 1, wherein the working portion comprises a blade, configured to cut tissue.

16. The method of claim 1, wherein the working portion comprises a sharpened tip, configured for piercing tissue.

17. The method of claim 1, wherein the working portion comprises a sharpened portion, configured to dissect tissue.

18. The method of claim 1, wherein the positions away from the position of mechanical contact are separated from the internal anatomical structure by a thickness of solid tissue.

19. The method of claim 18, wherein the thickness of solid tissue is at least 1 cm thick.

20. The method of claim 1, wherein the positions away from the position of mechanical contact are outside an exterior surface of an organ comprising the internal anatomical structure.

21. The method of claim 1, wherein the positions away from the position of mechanical contact are external to a cardiovascular lumen.

22. A method of modifying and using a medical implement having a designated use, the method comprising:
attaching at least one electrode to the medical implement;
using said medical implement for its designated use, while the at least one electrode transmits and/or receives an electrical field modified by intersection with tissue;
guiding movement of the medical implement, based on an image of the tissue reconstructed based on measurements of the electrical field transmitted and/or received by the at least one electrode.

23. The method of claim 22, wherein the at least one electrode transmits, and at least one additional measuring electrode is provided, positioned to sense changes in the electrical field during the using said medical implement.

24. The method of claim 22, wherein the at least one electrode receives, and at least one additional transmitting electrode is provided, positioned to transmit the electrical field during the using said medical implement.

25. The method of claim 22, comprising: moving the medical implement toward a portion of the imaged tissue, wherein the movement is guided by the image and a position of the medical implement relative to the image.

26. A system augmenting a medical implement, comprising:
the medical implement, wherein the medical implement comprises a body terminating in a rigid distal end, said rigid distal end being at least 5 cm in length, and at least one electrode within 3 cm from a distal-most tip of said rigid distal end; and
circuitry configured to transmit and/or receive electrical signals via the at least one electrode and provide an image of tissue adjacent said tip therefrom.

27. An imaging system comprising:
at least one electrode configured for attachment to a tool surface, or attached to a tool surface;
wherein said electrode is not used for a medical interaction with a body part;
signal circuitry configured to send and/or receive electrical signals from said electrode; and
reconstruction circuitry configured to reconstruct an image from said signals, said image including a representation of said tool based on said signals.
